SEQUENCE LISTING (18 sequences)

SEQ ID NO: 1 - DNA, 20 bp, Artificial Sequence
  Synthetic primer
gcttttgcaagctaggtccc

SEQ ID NO: 2 - DNA, 20 bp, Artificial Sequence
  Synthetic primer
tttctgatggggcgtaccac

SEQ ID NO: 3 - DNA, 20 bp, Artificial Sequence
  Synthetic primer
cagcctggacgagctggtgg

SEQ ID NO: 4 - DNA, 20 bp, Artificial Sequence
  Synthetic primer
tgaccagttggggttcacat

SEQ ID NO: 5 - DNA, 21 bp, Artificial Sequence
  Synthetic primer
cctccgtgctttttgaactcc

SEQ ID NO: 6 - DNA, 21 bp, Artificial Sequence
  Synthetic primer
catagccaaaggtcgttcctc

SEQ ID NO: 7 - DNA, 20 bp, Artificial Sequence
  Synthetic primer
agatctcgtcgatttctggc

SEQ ID NO: 8 - DNA, 20 bp, Artificial Sequence
  Synthetic primer
gtgcttgtcaccttgcaaaa

SEQ ID NO: 9 - DNA, 20 bp, Artificial Sequence
  Sunthetic primer
gagagcaaggaatcagccag

SEQ ID NO: 10 - DNA, 20 bp, Artificial Sequence
  Synthetic primer
gtgcttttcccagattcacc

SEQ ID NO: 11 - DNA, 20 bp, Artificial Sequence
  Synthetic primer
acagggacttgaatcgggtc

SEQ ID NO: 12 - DNA, 21 bp, Artificial Sequence
  Synthetic primer
tggtaaagtgggacggagttg

SEQ ID NO: 13 - DNA, 24 bp, Artificial Sequence
  Synthetic primer
cctccctgagtacatacaatgacc

SEQ ID NO: 14 - DNA, 24 bp, Artificial Sequence
  Synthetic primer
gtagtagcacctggtcttgctctt

SEQ ID NO: 15 - DNA, 21 bp, Artificial Sequence
  Synthetic primer
cgtgaatcttggctgtaaact

SEQ ID NO: 16 - DNA, 20 bp, Artificial Sequence
  Synthetic sequence
gtccgtggctctcttattct

SEQ ID NO: 17 - DNA, 19 bp, Artificial Sequence
  Synthetic primer
accgtctgtcgcttcactg

SEQ ID NO: 18 - DNA, 20 bp, Artificial Sequence
  Synthetic primer
ccactttatctgccgcttgc

(19) United States
(12) Patent Application Publication
(10) Pub. No.: US 2022/0054591 A1
(43) Pub. Date: Feb. 24, 2022

(54) METHODS OF TREATING OF INFLAMMATORY BOWEL DISEASE AND PARASITE INFECTION

(71) Applicant: President and Fellows of Harvard College, Cambridge, MA (US)

(72) Inventors: Wendy Garrett, Brookline, MA (US); Michael Howitt, Jamaica Plain, MA (US)

(21) Appl. No.: 17/390,176

(22) Filed: Jul. 30, 2021

Related U.S. Application Data
(62) Division of application No. 16/074,934, filed on Aug. 2, 2018, now abandoned.
(60) Provisional application No. 62/290,734, filed on Feb. 3, 2016.

(51) Int. Cl. A61K 38/20; A61K 39/39; A61K 39/02; A61P 33/00; A61P 1/00; A61K 9/00; C07K 14/47

(52) U.S. Cl. CPC A61K 38/20; A61K 39/39; A61K 39/02; Y02A 50/30; A61P 1/00; A61K 9/0053; C07K 14/473; A61P 33/00

(57) ABSTRACT

Described herein are methods for the induction of a T_{H}2 immune response and for the treatment and/or prevention of diseases associated with pathological immune responses and parasitic infection.

### RELATED APPLICATIONS

This application is a divisional of U.S. Ser. No. 16/074,934, filed Aug. 2, 2018, which is a § 371 national-stage application based on PCT/US17/016447, filed Feb. 3, 2017 which claims the benefit of priority to U.S. Provisional Patent Application Ser. No. 62/290,734, filed Feb. 3, 2016, each of which is hereby incorporated by reference in their entirety.

### GOVERNMENT INTEREST

This invention was made with Government support under National Institutes of Health Grants F32DK098826, R01 CA154426 and R01 GM099531. The Government has certain rights in the invention.

### BACKGROUND

Inflammatory bowel disease is group of inflammatory conditions of the colon and small intestine that cause over 50,000 deaths annually. The causes of inflammatory bowel disease are complex, and contributing factors may include diet, genetics, and the composition of an individual's gut microflora. Medical treatment is largely based on a factors specific to an individual.

Crohn's disease (CD) and ulcerative colitis (UC) are among the most common forms of inflammatory bowel disease. Both CD and UC are inflammatory diseases, but while UC is localized to the colon, Crohn's disease can affect any part of the gastrointestinal tract, from mouth to anus. Neither CD nor UC are currently medically curable, and current treatments range from surgical removal of parts of the intestine to administration of anti-inflammatory and/or immunosuppressive drugs. Unfortunately, current treatments for CD and UC are often ineffective and can result in significant side effects.

Parasitic diseases affect hundreds of millions of individuals, mostly in developing countries, where people are particularly susceptible to parasitic infection due to contaminated food and water and inadequate sanitation. The most common treatment for parasitic infection are antiparasitic drugs, such as albendazole and mebendazole. However, such treatments can be ineffective and repeated administration of such drugs can leave to drug resistance in the parasite populations.

Thus, there is a continuing need for new methods and compositions for the treatment of inflammatory bowel disease and parasitic diseases.

### SUMMARY

In certain aspects, provided herein are methods and compositions for inducing a type 2 helper T cell (T_{H}2) immune response in a subject comprising administering to the subject an agent that enhances the taste-chemosensory signaling pathway in a tuft cells. In certain aspects, provided herein are methods and compositions for treating and/or preventing an inflammatory bowel disease and/or a parasitic infection in a subject comprising administering to the subject an agent that enhances the taste-chemosensory signaling pathway in a tuft cells. In some embodiments, provided herein are methods and compositions to protect, repair or regenerate the intestinal epithelium which has been damaged or depleted or has potential to be damaged or depleted as a result of inflammatory bowel disease (e.g., ulcerative colitis, Crohn's disease). In some embodiments, the agent enhances the activity and/or expression of Trpm5, PLCB2 or gustducin. In some embodiments, the agent induces expression of IL-25 by the tuft cells and/or IL-13 by the subject.

In some aspects, provided herein are methods of inducing IL-25 expression by a tuft cell comprising contacting the tuft cell with an agent that enhances the taste-chemosensory signaling pathway in a tuft cells. In some embodiments, the tuft cell is contacted with the agent in vitro. In some embodiments, the tuft cell is administered to a subject after being contacted with the agent. In some embodiments, the tuft cell is isolated from the subject prior to being contacted with the agent. In some embodiments, the tuft cell is contacted with the agent in vivo.

In some embodiments of the methods provided herein, the agent is a small molecule agonist of Trpm5, PLCB2 or gustducin. In some embodiments, the agent is an antibody or antigen binding fragment thereof with binding specificity for Trpm5, PLCB2 or gustducin. In some embodiments, the agent comprises a nucleic acid (e.g., an mRNA or an expression vector) that encodes Trpm5, PLCB2 or gustducin.

In some embodiments of the methods provided herein, the agent activates a taste receptor. In some embodiments, the agent is a taste receptor ligand. In some embodiments, the agent is an antibody or antigen binding fragment thereof with binding specificity for the taste receptor. In some embodiments, the agent is a small molecule agonist of the taste receptor.

In some embodiments of the methods provided herein, the subject has or is predisposed to a disease associated with a pathological immune response (e.g., inflammatory bowel disease) In some embodiments, the subject has or is predisposed to a protozoan infection and/or to a parasitic worm infection.

### BRIEF DESCRIPTION OF THE DRAWINGS

FIG. 1 includes eight panels (Panels A-H) showing that symbiotic protozoa or helminths increase intestinal tuft cell abundance. Panel A is a bar graph showing DCLK1⁺ tuft cell frequency in small intestine (SI) from wild-type mice (WT) bred in-house (BIH) and at Jackson laboratories (JAX). Panel B shows H&E-stained SI sections from WT (BIH) and WT (JAX) scale bar, 50 μm (left) and higher magnification from WT (BIH), scale bar, 20 μm (right). Panel C is an scanning electron micrograph of protozoa isolated from WT (BIH) mice, scale bar, 4 μm. Panel D is a bar graph showing T. muris abundance in stool DNA (T. muris 28S rRNA relative to Eubacteria 16S rRNA) by qPCR; not detectable (ND). Panel E is a representative SI images from uninfected and T. muris colonized mice and Panel F is a bar graph showing tuft cell frequency. Panel G is a representative SI images from uninfected and helminth colonized mice and Panel H shows tuft cell frequency. Scale bars 100 μm in Panel E and Panel G. Each symbol represents an individual mouse and all data are representative of two (Panels D, F and H) or three (Panel A) independent experiments. T. muris infection was 17 days in (Panels E and F). In (Panles G and H) Hp infection was 21 days, Ts infection was 15 days, Nb infection was 8 days. Data plotted as mean with s.d. with ****P<0.0001, ***P=0.0001 calculated with one-way ANOVA or Mann-Whitney test.

FIG. 2 includes 4 panels (Panels A-D) showing tuft cell frequency is equal when using the markers DCLK1 and Gfilb. Panel A shows micrographs of small intestine from Gfilb^{EGFP/+} mice bred in-house (BIH). Scale bars 50 μm. Panel B is a representative flow plot of the epithelium from the distal small intestine of Gfilb^{EGFP/+} (BIH) mice. Panel C is a bar graphing showing expression data when tuft cells and non-tuft cell epithelial cells from Gfilb^{EGFP/+} mice were sorted by FACS and DCLK1 expression was determined by RT-qPCR. Data represent two independent experiments. Panel D is a bar graph showing the frequency of tuft cells (Gfi1b-GFP⁺) in the total epithelium of Gfilb^{EGFP/+} (BIH) mice as determined by flow cytometry. Symbols represent data from individual mice and are reflective of 5 experiments.

FIG. 3 shows feeding the cecal contents from WT (BIH) mice to WT (JAX) mice increases tuft cell abundance. Specifically, representative micrographs of the distal small intestine from WT (BIH) mice, WT (JAX) mice, and WT (JAX) mice 3 weeks after feeding the cecal contents obtained from WT (BIH) mice. Scale bars 100 μm. Data represent two independent experiments with 2-5 mice per group.

FIG. 4 includes two panels showing metronidazole treatment of WT (BIH) mice reduces Tritrichomonas muris levels below the limit of detection in stool and concomitantly reduces tuft cell frequency in the epithelium. Panel A is a bar graph of quantitative PCR (qPCR) comparing T. muris 28S rRNA levels relative to eubacteria 16S rRNA from stool DNA isolated from WT (BIH) mice given 2.5 g/L metronidazole in their drinking water or control mice; not detectable (ND). Panel B is a bar graph showing corresponding tuft cell frequency in either control or metronidazole-treated mice. **P=0.0015, Mann-Whitney test. Data represent two independent experiments with 2-4 mice per group.

FIG. 5 shows tritrichomonas colonizes germ-free mice and increases tuft cell abundance. Specifically, representative micrographs of the distal small intestine from germ-free C57BL/6 mice and germ-free C57BL/6 mice colonized with Tritrichomonas muris for 21 days. Scale bars 100 μm. Data are representative of 5 mice per group.

FIG. 6 is a simplified model of taste-chemosensation highlighting key taste-chemosensation effectors: gustducin, PLCβ2, and Trpm5.

FIG. 7 includes 9 panels (Panels A-I) showing that tuft cells influence type 2 immunity via Trpm5. Panel A is three bar graphs showing Gustducin, PLCβ2, and Trpm5 expression in sorted tuft cells compared to the non-tuft cell epithelium. Panel B shows representative images of T. muris (Tm) colonized WT and Gustducin^{-/-} mice and tuft cell frequencies. Panel C shows representative image from Trpm5^{eGFP} mice. Panel D shows representative image of T. muris colonized Trpm5−/− mice and tuft cell frequencies. Scale bars 50 μm (Panel B, C, and D). Panel E shows representative flow cytometry plots of IEC from uninfected (left) or T. muris colonized (right) WT (Gfilb^{EGFP/+}) (top) and Trpm5^{-/-} (Gfilb^{EGFP/+} Trpm5^{-/-}) (bottom) mice and Panel F is a bar graph showing tuft cell frequency. Panel G shows Goblet cells in SI sections stained with alcian blue/nuclear red in uninfected WT and T. muris colonized WT and Trpm5^{-/-} mice and Panel H shows goblet cell frequency. Panel I is a bar graph showing eosinophil frequency in the distal SI lamina propria (LP) of uninfected and T. muris colonized WT and Trpm5^{-/-} mice. Scale bars, 50 μm; each symbol represents an individual mouse and all data are representative of at least three independent experiments. Data plotted as mean with s.d.; ****P<0.0001; ***P=0.0001; **P<0.01; not significant (ns) calculated with one-way ANOVA, Kruskal-Wallis, or Mann-Whitney tests.

FIG. 8 includes 2 panels (Panels A and B) showing Trpm5-GFP⁺ cells in the distal small intestine are restricted to the epithelium and colocalize with DCLK1. Panel A is a representative image of the distal small intestine of Tritrichomonas muris colonized Trpm5^{eGFP} mice. Scale bar 100 μm. Panel is a plot showing the percentage of GFP⁺ cells in the distal small intestine from Trpm5^{eGFP} mice that are DCLK1⁺ EpCAM⁺ (tuft cells). Data represent 7 mice.

FIG. 9 includes 3 panels (Panels A-C) showing data measuring Tritrichomonas muris and Heligmosomoides polygyrus colonization. Panel A is a plot showing data after T. muris was counted with a hemocytometer and correlated with Ct values obtained by qPCR. A semi-log regression curve was fit, R²=0.998. Panel B is a bar graph showing the determination of T. muris abundance in the distal small intestine of WT, Trpm5^{-/-}, and Gustducin^{-/-} 10-18 mice per group. Panel C is a bar graph showing H. polygyrus counts from the distal small intestine of WT and Trpm5^{-/-} mice colonized for 36 days. **P=0.0087, Mann-Whitney test. Data represent 6 mice in each group and reflect 2 independent experiments.

FIG. 10 includes 6 panels (Panels A-F) showing that tuft cells express IL-25 and elicit ILC2s, in a Trpm5 dependent manner, in response to symbiotic protozoa. Panel A is a bar graph showing Il25 expression from sorted tuft cells. Panel B is plot showing WT (closed circles) and Trpm5^{-/-} (open circles) mice were colonized with T. muris for 3, 7, 12, and 42 days. At each time point, epithelial cell Il25 expression was measured (purple line) and T. muris colonization was quantified. Panel C is a bar graph showing the frequency of IL17RB⁺ (IL-25R) ILC2s in the distal SI LP of uninfected WT and WT and Trpm5^{-/-} mice colonized with T. muris for 12 days. Panel D is a bar graph showing Eosinophil frequency in the distal SI lamina propria of uninfected WT or T. muris colonized Trpm5^{-/-} mice i.p. injected with IL-25 or PBS control. Panel E is a bar graph showing tuft cell frequencies and Panel F shows flow plots of epithelial cells isolated from Trpm5^{-/-} mice i.p. injected with IL-25 or PBS. Each symbol in C, D, and E represents an individual mouse and all data are representative of three independent experiments. Data plotted as mean with s.d.; ***P<0.001; **P<0.01 calculated with Kruskal-Wallis or Mann-Whitney tests.

FIG. 11 includes 2 panels (Panels A and B) showing 11-33, TSLP, Il17RB expression in tuft cells. Epithelial cells from Gfilb^{EGFP/+} mice were sorted into tuft cell and non-tuft cell fractions. Panel A is a bar graph showing expression of Il-33 and TSLP determined by RT-qPCR. Panel B is a bar graph showing expression of Il17RB determined by RT-qPCR. ***P=0.0006, **P=0.0043, Mann-Whitney test. Data represent three independent experiments.

FIG. 12 includes 4 panels (Panels A-D) showing innate lymphoid cells and IL-13 increase tuft cells in organoids and the small intestine. Panel A shows differential interference contrast (DIC), fluorescent, and merged images of small intestinal organoids generated from Gfilb^{EGFP/+} mice, scale bars, 25 μm. Panel B is a bar graph showing GFP⁺ tuft cell abundance by flow cytometry of WT and Trpm5^{-/-} organoids treated with recombinant IL-13 or IL-25. Panel C shows Representative images of SI from WT, Stat6^{-/-}, Rag2^{-/-}, and Rag2^{-/-} Il2rγ^{-/-} mice colonized with T. muris and Panel D shows tuft cell frequency. Scale bars, 100 μm. Each symbol represents an individual mouse and all data are representative of (Panel D) two or (Panel B) three independent experiments. Data plotted as mean with s.d. with ****P<0.0001; not significant (ns) calculated with one-way ANOVA or Mann-Whitney tests.

FIG. 13 includes 3 panels (Panels A-C) showing 11-13 increases tuft cell abundance in both WT and Trpm5^{-/-} organoids. Panel A shows representative flow cytometry plots of WT (Gfilb^{EGFP/+}) and Trpm5^{-/-} (Gfilb^{EGFP/+} Trpm5^{-/-}) organoids treated with 11-13 and Il-25. Expression of DCLK1 (Panel B) and Trpm5 (Panel B) in organoids treated with 11-13 is shown. Data are plotted as mean with s.d. and are representative of three independent experiments.

FIG. 14 is a bar graph showing tritrichomonas muris equivalently colonizes WT, Stat6−/−, Rag2−/−, and Rag2−/−IL2rγ−/− mice. Small intestinal contents were analyzed by qPCR for T. muris abundance. T. muris abundance was not significantly (ns) different between mice as calculated by Ordinary one-way ANOVA. Data are representative of two independent experiments, 5-10 mice per group.

FIG. 15 depicts a model in accordance with the invention. (1) Tuft cells respond to lumenal parasites and utilize Trpm5 dependent upstream signaling pathways. (2) In response to parasite colonization, tuft cells produce IL-25 which expands and activates ILC2s (3) ILC2s then produce type 2 cytokines such as IL-13 that signal back to the epithelium to increase both goblet cells and tuft cells. (4) ILC2s control eosinophila through production of IL-5 and IL-13. Not wishing to be bound by any particular theory, it is proposed that IL-25 released by tuft cells may increase eosinophils through the accumulation and activation of ILC2s.

FIG. 16 depicts a plot showing gating strategy for eosinophils. Cells were isolated from the distal small intestine lamina propria and gated on CD45⁺PI⁻ cells. Eosinophils were selected as CD11b⁺MHCII⁻SiglecF⁺ and SSC^{hi}.

FIG. 17 depicts a plot showing gating strategy for ILC2s. Cells from the distal small intestinal lamina propria were isolated and gated on viable CD45⁺ cells. Il-25-responsive ILC2s were further selected as Lin-IL7Rα⁺KLRG1⁺IL17RB⁺.

### DETAILED DESCRIPTION

### General

In certain aspects, provided herein are methods related to the administration of an agent to enhance the taste-chemosensory signaling pathway in tuft cells. As disclosed herein, the disruption of chemosensory signaling (e.g., via loss of Trpm5) abrogates expansion of tuft cells, goblet cells, eosinophils, and type-2 innate lymphoid cells (ILC2s) during parasite colonization. Tuft cells are the primary source of the parasite-induced cytokine, IL-25, which indirectly induces tuft cells expansion by promoting IL-13 production by ILCs. As described herein, intestinal tuft cells are critical sentinels in the gut epithelium that promote type-2 immunity in response to intestinal parasites. As such, in some embodiments the methods disclosed herein are useful in treating or preventing diseases associated with a T_{H}1 and/or T_{H}17 immune response (e.g., inflammatory bowel disease), and infections responsive to a T_{H}2 immune response (e.g., parasitic infection) by inducing a type 2 helper T cell (T_{H}2) immune response in a subject. In some embodiments, provided herein are methods and compositions to protect, repair or regenerate the intestinal epithelium which has been damaged or depleted or has potential to be damaged or depleted as a result of inflammatory bowel disease (e.g., ulcerative colitis, Crohn's disease). In certain embodiments, the instant invention relates to a method of inducing IL-25 expression by a tuft cell comprising contacting the tuft cell with an agent that enhances the taste-chemosensory signaling pathway in a tuft cells. Such cells can be, for example, induced to express IL-25 ex vivo and then transplanted into a subject to treat or prevent an inflammatory disease and/or a parasitic infection.

In certain embodiments, the agent administered in the methods disclosed herein is an agent that enhances the activity or expression of Trpm5, PLCB2, and/or gustducin, such as a small molecule, an antibody or a nucleic acid that enhances the activity or expression of Trpm5, PLCB2 and/or gustducin. In some embodiments the agent that administered according to the methods described herein activates a taste receptor on the tuft cells. In some embodiments, the agent is a taste receptor ligand, an antibody or antigen binding fragment with binding specificity for the taste receptor, or a small molecule agonist of the taste receptor.

### Definitions

For convenience, certain terms employed in the specification, examples, and appended claims are collected here.

The articles “a” and “an” are used herein to refer to one or to more than one (i.e., to at least one) of the grammatical object of the article.

As used herein, the term “administering” means providing an agent or composition to a subject, and includes, but is not limited to, administering by a medical professional and self-administering.

The terms “agent” are used herein to denote a chemical compound, a small molecule, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments. An “isolated antibody,” as used herein, refers to an antibody which is substantially free of other antibodies having different antigenic specificities. An isolated antibody may, however, have some cross-reactivity to other, related antigens.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

As used herein, an “effective amount” is an amount effective in treating or preventing a disease associated with a pathological immune response, including, for example, inflammatory bowel disease.

As used herein, the term “enhance” refers to improve, increase, amplify, multiply, elevate, raise, and the like.

As used herein, the term “humanized antibody” refers to an antibody that has at least one CDR derived from a mammal other than a human, and a FR region and the constant region of a human antibody. A humanized antibody is useful as an effective component in a therapeutic agent according to the present invention since antigenicity of the humanized antibody in human body is lowered.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination thereof, which (1) is not associated with the cell in which the “isolated nucleic acid” is found in nature, or (2) is operably linked to a polynucleotide to which it is not linked in nature.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

As used herein, the term “subject” means a human or non-human animal selected for treatment or therapy. In certain embodiments of the methods described herein, the subject is a human subject.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

“Treating” a disease in a subject or “treating” a subject having a disease refers to subjecting the subject to a pharmaceutical treatment, e.g., the administration of a drug, such that at least one symptom of the disease is decreased or prevented from worsening.

### Trpm5

In certain embodiments, the methods provided herein relate to agents that enhance the expression and/or activity or Trpm5. As used herein, the term “Trpm5” or “Trpm5 protein” refers to the transient receptor potential cation channel subfamily M member 5 protein, which is also known as the long transient receptor potential channel 5 protein. In humans, Trpm5 is encoded by the TRPM5 gene. Exemplary human Trpm5 mRNA and protein sequences are provided at NCBI accession numbers NM_014555.3 and NP_064673.2, respectively, each of which is hereby incorporated by reference.

### PLCB2

In certain embodiments, the methods provided herein relate to agents that enhance the expression and/or activity or PLCB2. As used herein, the term “PLCB2” or “PLCB2 protein” refers to the 1-Phosphatidylinositol-4,5-bisphosphate phosphodiesterase beta-2 protein. In humans, PLCB2 is encoded by the PLCB2 gene. Exemplary human PLCB2 mRNA and protein sequences are provided at NCBI accession numbers NM_001284297.1 and NP_001271226.1, respectively, each of which is hereby incorporated by reference.

### Gustducin

In certain embodiments, the methods provided herein relate to agents that enhance the expression and/or activity or Gustducin. Gustducin is a G protein associated with taste and the gustatory system found in certain taste receptor cells. Gustducin is a heterotrimeric protein composed of the protein products of the GNAT3, GNB1 and GNG13 genes. Exemplary human GNAT3 mRNA and protein sequences are provided at NCBI accession numbers NM_01102386.2 and NP_001095856.1, respectively, each of which is hereby incorporated by reference. Exemplary human GNB1 mRNA and protein sequences are provided at NCBI accession numbers NM_001282538.1 and NP_001269467.1, respectively, each of which is hereby incorporated by reference. Exemplary human GNG13 mRNA and protein sequences are provided at NCBI accession numbers NM_016541.2 and NP_057625.1, respectively, each of which is hereby incorporated by reference.

### Taste-Receptors

In certain embodiments, the methods provided herein relate to agents that enhance the activity and/or expression of a taste receptor. For example, in some embodiments, the agent is a small molecule agonist of a taste receptor, a taste receptor ligand, or an antibody or antigen binding fragment thereof with binding specificity for the taste receptor. In some embodiments, the taste receptor is a human taste receptor. In some embodiments, the taste receptor is expressed on a tuft cell.

In some embodiments, the agent enhances the activity or expression of any human taste receptor expressed on a tuft cell. In some embodiments, the agent enhances the activity and/or expression of a Type 1 taste receptor. In some embodiments, the agent enhances the activity and/or expression of a Type 2 taste receptor. In some embodiments, the agent enhances the activity or expression of TAS1R1. In some embodiments, the agent enhances the activity or expression of TAS1R2. In some embodiments, the agent enhances the activity or expression of TAS1R3. In some embodiments, the agent enhances the activity or expression of TAS1R4. In some embodiments, the agent enhances the activity or expression of TAS2R1. In some embodiments, the agent enhances the activity or expression of TAS2R3. In some embodiments, the agent enhances the activity or expression of TAS2R4. In some embodiments, the agent enhances the activity or expression of TAS2R5. In some embodiments, the agent enhances the activity or expression of TAS2R7. In some embodiments, the agent enhances the activity or expression of TAS2R8. In some embodiments, the agent enhances the activity or expression of TAS2R9. In some embodiments, the agent enhances the activity or expression of TAS2R10. In some embodiments, the agent enhances the activity or expression of TAS2R12. In some embodiments, the agent enhances the activity or expression of TAS2R13. In some embodiments, the agent enhances the activity or expression of TAS2R14. In some embodiments, the agent enhances the activity or expression of TAS2R15. In some embodiments, the agent enhances the activity or expression of TAS2R16. In some embodiments, the agent enhances the activity or expression of TAS2R18. In some embodiments, the agent enhances the activity or expression of TAS2R19. In some embodiments, the agent enhances the activity or expression of TAS2R20. In some embodiments, the agent enhances the activity or expression of TAS2R22. In some embodiments, the agent enhances the activity or expression of TAS2R23. In some embodiments, the agent enhances the activity or expression of TAS2R30. In some embodiments, the agent enhances the activity or expression of TAS2R31. In some embodiments, the agent enhances the activity or expression of TAS2R33. In some embodiments, the agent enhances the activity or expression of TAS2R36. In some embodiments, the agent enhances the activity or expression of TAS2R37. In some embodiments, the agent enhances the activity or expression of TAS2R38. In some embodiments, the agent enhances the activity or expression of TAS2R39. In some embodiments, the agent enhances the activity or expression of TAS2R40. In some embodiments, the agent enhances the activity or expression of TAS2R41. In some embodiments, the agent enhances the activity or expression of TAS2R42. In some embodiments, the agent enhances the activity or expression of TAS2R43. In some embodiments, the agent enhances the activity or expression of TAS2R44. In some embodiments, the agent enhances the activity or expression of TAS2R45. In some embodiments, the agent enhances the activity or expression of TAS2R46. In some embodiments, the agent enhances the activity or expression of TAS2R47. In some embodiments, the agent enhances the activity or expression of TAS2R48. In some embodiments, the agent enhances the activity or expression of TAS2R49. In some embodiments, the agent enhances the activity or expression of TAS2R50. In some embodiments, the agent enhances the activity or expression of TAS2R60.

### Nucleic Acids

In some embodiments, the methods provided herein relate to the administration of a nucleic acid encoding one or more of the proteins described herein (e.g., Trpm1, PLCB2, Gustducin and/or a taste receptor) to a subject and/or to a cell (e.g., a tuft cell). In some embodiments, the nucleic acid is an mRNA molecule and/or a vector encoding an mRNA molecule. In some embodiments, the nucleic acid is linked to a promoter and/or other regulatory sequences. In some embodiments, the nucleic acid comprises a sequence that is at least about 80%, 85%, 90%, 95%, 98%, 99% or 100% identical to a nucleotide sequence provided herein.

In some embodiments, the nucleic acids described herein (e.g., those encoding a protein of interest or functional homolog thereof, or a nucleic acid intended to enhance the production of a protein described herein) can be delivered to cells in culture, ex vivo, and in vivo. The delivery of nucleic acids can be delivered by any technique known in the art including, but not limited to, viral mediated gene transfer and liposome mediated gene transfer. Polynucleotides can be administered in any suitable formulations known in the art. These can be as virus particles, as naked DNA, in liposomes, in complexes with polymeric carriers, etc.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W.H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

One or more polypeptide (e.g., a Trpm5 protein, PLCB2 protein or gustducin protein) or nucleic acid of interest may be encoded by a single nucleic acid. Alternatively, separate nucleic acids may encode different protein or nucleic acids of interest. Different species of nucleic acids may be in different forms; they may use different promoters or different vectors or different delivery vehicles. Similarly, the same protein or nucleic acid of interest may be used in a combination of different forms.

In certain embodiments, the instant invention relates to vectors that contain the isolated nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

### Antibodies

In certain embodiments, the methods provided herein relate to the delivery and/or use of antibodies and antigen binding fragments thereof that bind specifically to Trpm5, PLCB2, gustducin or a taste receptor described herein. Such antibodies can be polyclonal or monoclonal and can be, for example, murine, chimeric, humanized or fully human.

Polyclonal antibodies can be prepared by immunizing a suitable subject (e.g. a mouse) with a polypeptide immunogen (e.g., a Trpm5, PLCB2, gustducin, or taste receptor polypeptide). The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal specific for Trpm5, PLCB2, gustducin or a taste receptor can be identified and isolated by screening a recombinant combinatorial immunoglobulin library (e.g., an antibody phage display library or an antibody yeast display library) with the appropriate polypeptide (e.g. a Trpm5, PLCB2, gustducin, or taste receptor polypeptide) to thereby isolate immunoglobulin library members that bind the polypeptide.

Additionally, recombinant antibodies specific for Trpm5, PLCB2, gustducin, or a taste receptor, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for Trpm5, PLCB2, gustducin or a taste receptor can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGx monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies described herein are able to bind to an epitope of Trpm5, PLCB2, gustducin or a taste receptor with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis.

### Small Molecules

In certain embodiments, the agent is a small molecule agonist of Trpm5, PLCB2, gustducin, or a taste receptor.

Agents (e.g., small molecules) useful in the methods described herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

### Formulations

As discussed herein, an agent described herein can be administered in any suitable formulation known in the art. Exemplary of routes of administration include oral administration, rectal administration, topical administration, inhalation (nasal) or injection. Administration by injection includes intravenous (IV), intramuscular (IM), intratumoral (IT) and subcutaneous (SC) administration. In certain preferred embodiments the agent is administered orally to the subject.

In some embodiments, the agent is delivered in a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the agent is delivered in a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Exemplary Methods of Treatment and Prevention of Diseases

Provided herein are methods of treatment or prevention of conditions and diseases that can be improved by enhancing the taste-chemosensory signaling pathway in tuft cells. The methods described herein can be used to treat any subject in need thereof. The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). In certain embodiments, the subject is human.

In certain aspects, provided herein are methods of inducing a type 2 helper T cell (T_{H}2) immune response in a subject comprising administering to the subject an agent that enhances the taste-chemosensory signaling pathway in a tuft cell. In certain aspects, provided herein are methods of inhibiting a type 1 helper T cell (T_{H}1) immune response in a subject comprising administering to the subject an agent that enhances the taste-chemosensory signaling pathway in a tuft cell. In certain aspects, provided herein are methods of inhibiting a type 17 helper T cell (T_{H}17) immune response in a subject comprising administering to the subject an agent that enhances the taste-chemosensory signaling pathway in a tuft cell. In certain embodiments, the agent induces expression of IL-25 by the tuft cells. In certain embodiments, the agent induces expression of IL-13 by the subject.

In certain embodiments, the subject has a disease or disorder associated with a pathological immune response (e.g., an inflammatory bowel disease), as well as any subject with an increased likelihood of acquiring a such a disease or disorder (e.g., predisposed). . In some embodiments, the subject has a damaged or depleted intestinal epithelium e.g., as a result of a pathological immune response, such as an inflammatory bowel disease.

In some embodiments, the disease or disorder is an inflammatory bowel disease (e.g., Crohn's disease, ulcerative colitis). In some embodiments, provided herein are methods of treating an inflammatory bowel disease. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In certain embodiments, the subject has or is predisposed to a protozoan infection. Exemplary protozoan infections include but are not limited to leishmaniasis, trichomoniasis, trypanosomiasis (Chagas disease and sleeping sickness), toxoplasmosis, malaria, giardiasis, cryptosporidiosis, babesiosis, primary amoebic meningoencephalitis, amoebiasis, dientamoebiasis, rhinosporidosis, sarcocystosis, cyclosporiasis, isosporiasis, blastocystosis, balantidiasis, and granulomatous amoebic encephalitis.

In certain embodiments, the subject has or is predisposed to a parasitic worm infection. Exemplary parasitic worms include but are not limited to coenurosis, diphyllobothriasis, echinococcosis, hymenolepiasis, taeniasis, cysticercosis, bertielliasis, sparganosis, schistosomiasis, clonorchiasis, fasciolosis, fasciolopsiasis, gnathostomiasis, metagonimiasis, paragonimiasis, opisthorchiasis, ascariasis, ancylostomiasis, angiostrongyliasis, baylisascariasis, filariasis, dracunculiasis, enterobiasis, halicephalobiasis, onchocerciasis, strongyloidiasis, thelaziasis, toxocariasis, trichinosis, trichuriasis, and elephantiasis.

In certain aspects, provided herein are methods of inducing IL-25 expression by a tuft cell comprising contacting the tuft cell with an agent that enhances the taste-chemosensory signaling pathway in a tuft cells. In some embodiments, the tuft cell is isolated from a subject (e.g., a subject in need thereof) prior to induction of IL-25 expression. In certain embodiments, the tuft cell is contacted with the agent in vitro or ex vivo. In certain embodiments, the tuft cell is administered to the subject after being contacted with the agent in order to induce a T_{H}2 immune response in the subject.

In certain aspects, provided herein are methods of regenerating damaged intenstinal epithelium resulting from inflammatory bowel disease (e.g., Crohn's disease, ulcerative colitis) comprising contacting the epithelium cell (e.g., tuft cell) with an agent that enhances the taste-chemosensory signaling pathway in the epithelium cell (e.g., tuft cell).

In certain aspects, provided herein are methods of repairing damaged intenstinal epithelium resulting from inflammatory bowel disease (e.g., Crohn's disease, ulcerative colitis) comprising contacting the epithelium cell (e.g., tuft cell) with an agent that enhances the taste-chemosensory signaling pathway in the epithelium cell (e.g., tuft cell).

In certain aspects, provided herein are methods of repairing depleted intenstinal epithelium resulting from inflammatory bowel disease (e.g., Crohn's disease, ulcerative colitis) comprising contacting an epithelium cell (e.g., tuft cell) with an agent that enhances the taste-chemosensory signaling pathway in the epithelium cell (e.g., tuft cell).

### EXEMPLIFICATION

The invention now being generally described will be more readily understood by reference to the following examples which are included merely for purposes of illustration of certain aspects and embodiments of the present invention, and are not intended to limit the invention in any way.

### Experimental Procedures

Mice—Wild-type C57BL/6J mice designated as bred in-house (BIH) were bred and housed in microisolator cages in the specific-pathogen-free (SPF) barrier facility at the Harvard T. H. Chan School of Public Health. Wild-type C57BL/6J designated as (JAX), Rag2^{-/-}, Gfilb^{EGFP/+} and Stat6^{-/-} mice were obtained from The Jackson Laboratory, Bar Harbor, Me. C57BL/6 Rag2^{-/-} Il2rγ^{-/-} mice were obtained from Taconic Biosciences, Germantown, N.Y. C57BL/6J Trpm5^{-/-}, gustducini^{-/-}, and Trpm5^{eGFP} mice were generously provided by Dr. Robert Margolskee (Monell Chemical Senses Center). C57BL/6J Gfilb^{eGFP/+}Trpm5^{-/-} mice were generated by breeding Gfilb^{eGFP/+} and Trpm5^{-/-} mice. Germ-free WT C57BL/6 mice were bred and maintained in vinyl positive pressure isolators within the Germ-free and Gnotobiotic core facilities at the Harvard Digestive Diseases Center at Brigham and Women's Hospital. Animal studies and experiments were approved and carried out in accordance with Harvard Medical School's Standing Committee on Animals and the National Institutes of Health guidelines for animal use and care. Helminth infections were carried out at Weill Cornell Medical School, Tufts University School of Medicine or Harvard T. H. Chan School of Public Health according to Institutional Animal Care and Use Committees (IACUC), and all experiments were performed according to the guidelines of the relevant institution.

Antibiotic treatment—Mice were treated with metronidazole (2.5 g/L) with 1% sucrose in the drinking water for 7 days. Control mice were given 1% sucrose water over the same time span. Fluid intake was monitored and metronidazole solution was changed 4 days after initiating antibiotic treatment.

Histology and Fluorescence microscopy —The small intestine was removed and divided into proximal and distal sections before fixation in 4% paraformaldehyde. The tissue was then embedded in paraffin and cut into 5 μm thick sections. For both histology and immunofluorescence, sections were initially deparaffinized and rehydrated. Hematoxylin and eosin (H&E) staining was performed using standard procedures. Goblet cells were identified by alcian blue/nuclear red staining and enumerated along the crypt-villus axis by quantitative microscopy. For immunofluorescence, heat-mediated antigen retrieval was performed in Tris-EDTA buffer 0.05% Tween-20 pH 9.0 for 20 minutes. Afterwards, the slides were washed in PBS and blocked in PBS containing 3% BSA, 3% donkey serum, 0.1% Triton X-100, 0.1% saponin for 1 hour at room temperature. Primary antibodies were incubated overnight at 4° C. and secondary antibodies were applied for 1.5 hours at room temperature. Primary antibodies included: rabbit anti-DCLK1 (1:250 dilution, ab37994, Abcam), mouse anti-E-Cadherin (1:400 dilution, 36/E-Cadherin, BD Biosciences) and DNA was labeled with DAPI (0.5 μg/ml). For colocalization of anti-DCLK1 and GFP, tissue was harvested from Gfilb^{EGFP/+} or Trpm5^{eGFP} mice and fixed as described above. The tissue was then incubated at 4° C. in PBS with 20% sucrose for 6 hours followed by PBS with 30% sucrose overnight prior to freezing in OCT compound. 8 μm frozen sections were cut and labeled with the following primary antibodies for Trpm5eGFP: anti-GFP (1:1500, ab13970), anti-DCLK1 (1:100 dilution, ab37994), DAPI (0.5 μg/ml), and either Phalloidin (1:400 dilution, Molecular probes) or APC-conjugated anti-EpCam (1:1200 dilution, clone G8.8, Biolegend). For localization of GFP and DCLK1 with Gfilb^{EGFP/+} the following antibody was used: anti-DCLK1 (1:100 dilution, ab37994) and DAPI (0.5 μg/ml). Images were captured with Nikon eclipse Ni-U microscope and processed with Nikon NIS-elements software.

Isolation and culture of Tritrichomonas muris-Isolation and culture of Tritrichomonas muris was performed using a modified protocol described by Saeki et al., Nippon Juigaku Zasshi. 45, 151-156 (1982). Briefly, cecal contents were harvested from WT C57BL/6J (BIH) mice passed through a 40 μm filter and washed three times in PBS. Trichomonads were further purified at the interface of a 40%/80% percoll (GE healthcare) gradient after centrifugation at 1000×g for 15 minutes without braking. The number and viability of the isolated T. muris was determined by counting with a hemocytometer. Approximately 5×10⁵ T. muris trophozoites were inoculated per ml of growth media, which consisted of Trichosel^{f}h broth (Becton Dickinson) suspended in cecal extract supplemented with 10% heat-inactivated horse serum, amphotericin B, gentamicin, penicillin, streptomycin, and vancomycin pH adjusted to 7.0. The culture of T. muris was then placed at 37° C. in an anaerobic cabinet.

Infection with protozoa and helminthes—Tritrichomonas muris was isolated and cultured as described above. The helminth maintenance and infection was performed as previously described for Heligmosmoides polygyrus (Lin et al., The Journal of Immunology. 185, 3184-3189 (2010)), Trichinella spiralis (Hotez et al., Journal of Clinical Investigation. 118, 1311-1321 (2008)), and Nippostrongylus brasiliensis (H.-E. Liang et al., Nat Immunol. 13, 58-66 (2011)). 5×10⁶ T. muris, 150 L3 H. polygyrus, or 500 T. spiralis muscle larvae were orally administered to mice. 500 L3 N. brasiliensis were subcutaneously injected into mice. Mice were infected and sacrificed according to the following schedule: T. muris were sacrificed 17 days post-infection, H. polygyrus 21 days post-infection, T. spiralis 15 days post-infection, and N. brasiliensis 8 days post-infection. For H. polygyrus counts, mice were inoculated with 150 L3 larvae and sacrificed 36 days later. The proximal small intestine was excised and worms were counted with a dissection microscope. For the Tritrichomonas muris time course, WT and Trpm5^{-/-} mice were infected with 5×10⁶ T. muris and then sacrificed 3, 7, 12, and 42 days later. Germ-free mice were inoculated with approximately 5×10⁶ T. muris for 21 days before excising the distal small intestine and processing the tissue for frozen sections a described above.

Scanning electron microscopy—Tritrichomonas muris was isolated from the cecal contents of WT (BIH) mice as described above and suspended in PBS. Protozoa were adhered to poly-_{L}-lysine coated coverslips and fixed in 2.5% glutaraldehyde in a 0.1 M cacodylate buffer, pH 7.2. Following 3 buffer rinses the protozoa were post-fixed for 30 min in 1% OsO4 in 0.1 M cacodylate buffer, dehydrated in a graded series of ethanol, and critical point dried with liquid CO2. T. muris was then sputter-coated with 5 nm platinum and examined with a Hitachi S-4800 field emission scanning electron microscope.

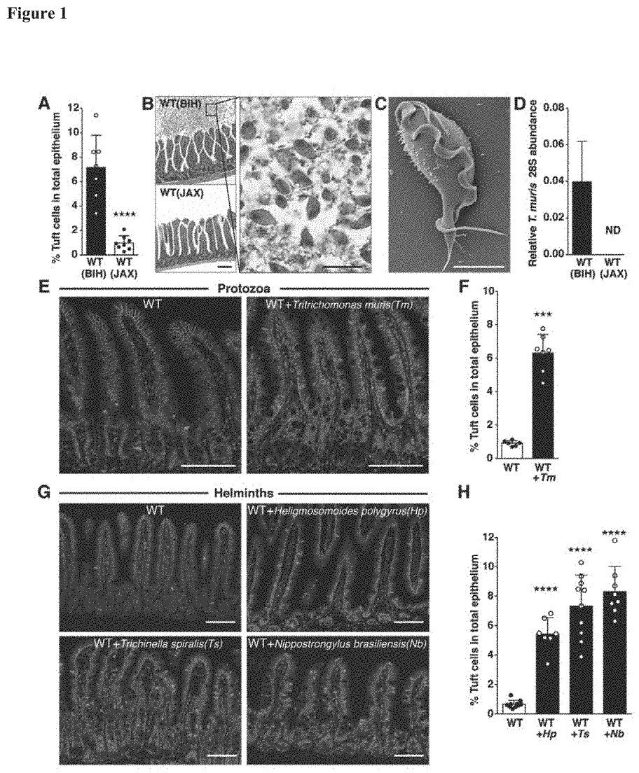

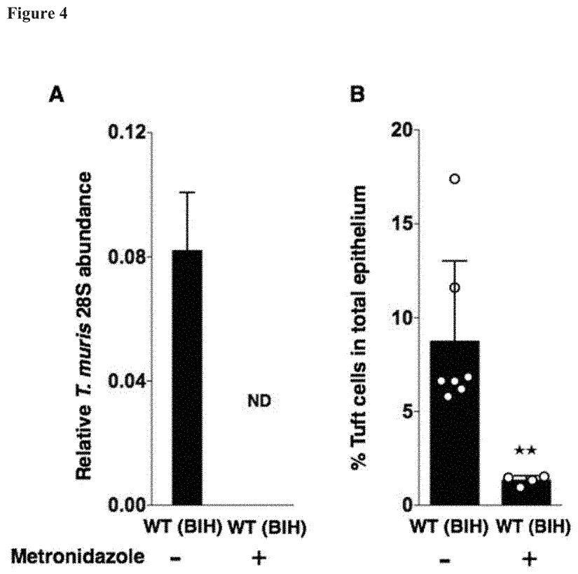

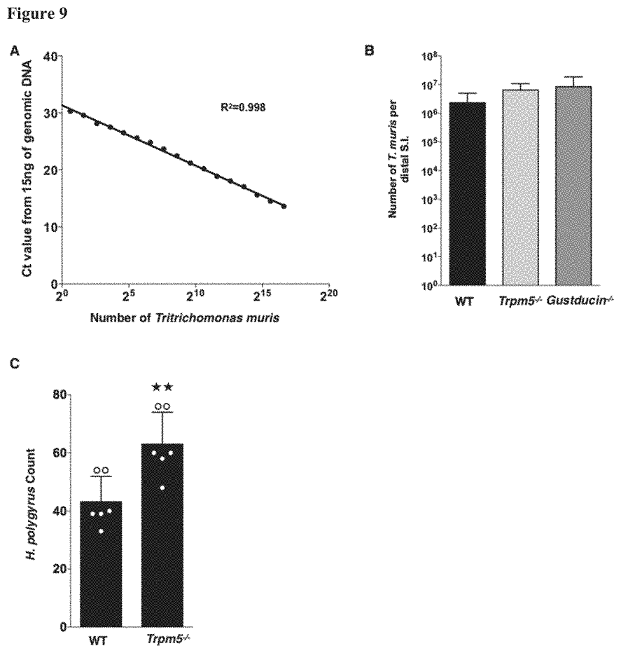

Tritrichomonas muris enumeration—The distal 10 cm of small intestine was removed and flushed with ice-cold sterile PBS using a 19-gauge feeding needle. The intestinal contents were then pelleted by centrifugation and stored at −20° C. Genomic DNA was isolated from the stool with QIamp Fast DNA Stool mini kit (Qiagen) according to the manufacturer's directions. To detect and enumerate Tritrichomonas muris, quantitative PCR (qPCR) was performed using KAPPA SYBR fast Universal PCR (KAPPA Biosystems) with the following primers recognizing T. muris 28S rRNA gene: 5′-GCTTTTGCAAGCTAGGTCCC-3′(SEQ ID NO. 1) and 5′-TTTCTGATGGGGCGTACCAC-3′ (SEQ ID NO. 2). To convert qPCR values into parasite numbers, T. muris trophozoytes were isolated and counted using a hemocytomer before extracting genomic DNA and analyzing by qPCR. These results were plotted on a standard curve and a regression analysis was performed to convert Ct values to parasite numbers (FIG. 9, Panel A). To determine T. muris abundance in stool as shown in FIG. 1, Panel D and FIG. 4, Panel A, DNA was isolated as described above. The T. muris 28S rRNA gene and Eubacteria 16S rRNA gene were amplified by qPCR and T. muris 28S relative abundance was calculated as 2^{-ΔCt(28S-16S)}.

Epithelial cell isolation and flow cytometry —The distal 10 cm of small intestine was removed and flushed as described above. The intestine was opened longitudinally and gently agitated at 4° C. in PBS, 2% FBS, 5 mM EDTA, 1 mM DTT for 10 min. The tissue was then transferred into prewarmed PBS, 2% FBS, 5 mM EDTA and rotated at 37° C. for 15 minutes followed by vigorous shaking to remove epithelial cells. This was repeated and epithelial cells from both fractions were combined and washed with PBS. The epithelium was then digested in DMEM containing 10% FBS, 0.5 units/ml Dispase II (StemCell Technologies), 50 μg/ml DNase (Roche) for 12 minutes at 37° C. The resulting solution was passed through 40 μm filters and washed with PBS, 2% FBS, 1 mM EDTA. The resulting single cell suspension was initially Fc blocked with anti-CD16/CD32 (clone 93, Biolegend) and then stained with the following antibodies: PacBlue-conjugated anti-CD45 (clone 30-F11, Biolegend), APC-conjugated anti-EpCam (clone G8.8, Biolegend). The cell viability was assessed by propidium iodide (PI) (Biolegend) staining.

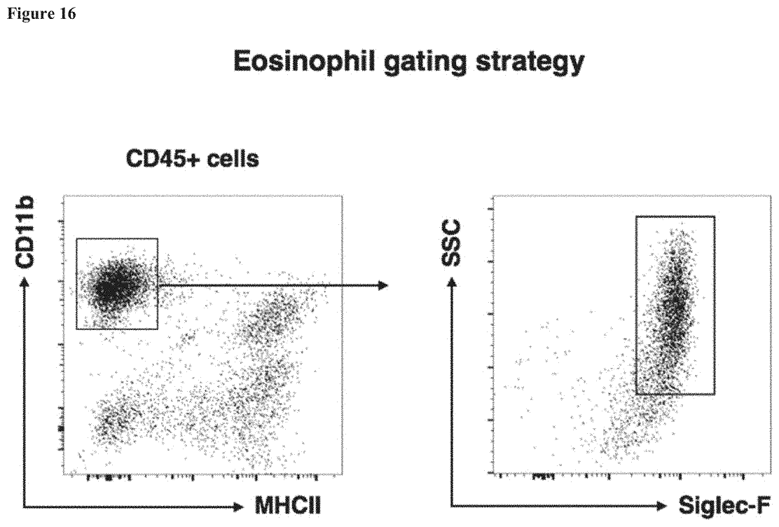

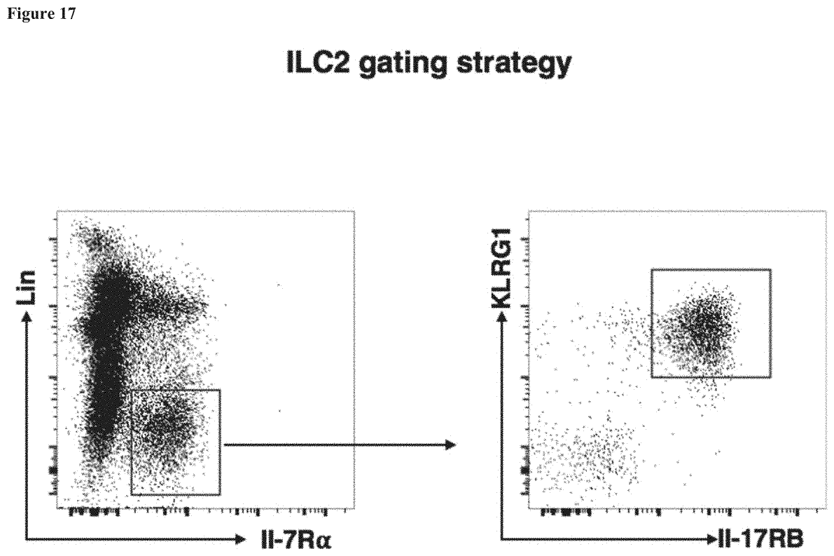

Lamina propria cell isolation and flow cytometry—The distal 10 cm of small intestine was collected and epithelial cells were removed as detailed above. Afterwards the tissue was minced into approximately 1 mm² sections and digested in RPMI 10% FBS, 0.25 mg/ml collagenase A (Roche), 0.1 units/ml Dispase II (StemCell Technologies), 50 μg/ml DNase (Roche) for 25 minutes followed by a second 40 minute digestion. The solution was passed through 40 μm filters and the resulting single-cell suspension was Fc blocked with anti-CD16/CD32 (clone 93, Biolegend) before staining with the following combination of antibodies (all antibodies are from Biolegend unless otherwise stated). For eosinophils: PacBlue-conjugated anti-CD45 (clone 30-F11), PE-Cy7-conjugated anti-I-A/I-E (clone M5/114.15.2), APC-conjugated anti-Siglec-F (clone E50-2440, 1D biosciences), APC-Cy7-conjugated anti-CDIlb (clone M1/70). PI (Biolegend) was used to exclude dead cells (gating strategy FIG. 16). For innate lymphoid cells, the following antibodies were used: Alexa Fluor® 488-conjugated anti-CD45 (clone 30-F11), PE-conjugated anti-IL17RB (clone 12-7361, eBioscience), PerCP-Cy5.5 conjugated anti-KLRG1 (clone 2F1/KLRG1), APC-conjugated anti-IL7Ra (clone A7R34) and lineage markers PacBlue-conjugated anti-CD3 (clone 17A2), PacBlue-conjugated anti-GR-1 (clone RB6-8C5), PacBlue-conjugated anti-CD1 b (clone M1/70), PacBlue-conjugated anti-B220 (clone RA3-6B2), PacBlue-conjugated anti-Ter119 (clone Ter-119), PacBlue-conjugated anti-CD4 (clone RM4-5), PaccBlue-conjugated anti-CD8a (clone 53-6.7), PaccBlue-conjugated anti-NK1.1 (clone PK136). After antibody staining dead cells were excluded with Live/Dead fixable yellow dead cell kit (Invitrogen) (gating strategy FIG. 17).

RNA isolation and RT-PCR for in vivo and vitro tuft cells—Epithelial cells from Gfi1b^{EGFP/+} mice were isolated and stained for Fluorescence Activated Cell Sorting (FACS) as previously detailed. Tuft cells were sorted based on GFP⁺EpCam⁺CD45⁻PI⁻ while the remaining epithelial cells were GFP-EpCam⁺CD45⁻PI⁻. RNA was then extracted from tuft cells and the remaining epithelium using RNeasy Micro Kit (Qiagen). Whole intestinal organoids were initially stored in RNAlater solution (Ambion) before extracting RNA using Qiazol (Qiagen) according to the manufacturer's directions. cDNA was synthesized using the iScript cDNA Synthesis Kit (Bio-Rad) and RT-qPCR was performed using the KAPA SYBR FAST Universal qPCR Kit (KAPA Biosystems). The following primers were used: DCLK1: 5′-CAGCCTGGACGAGCTGGTGG-3′ (SEQ ID NO. 3) and 5′-TGACCAGTTGGGGTTCACAT-3′ (SEQ ID NO. 4), Trpm5: 5′-CCTCCGTGCTTTTTGAACTCC-3′ (SEQ ID NO. 5) and 5′-CATAGCCAAAGGTCGTTCCTC-3′ (SEQ ID NO. 6), PLCβ2: 5′-AGATCTCGTCGATTTCTGGC-3′ (SEQ ID NO. 7) and 5′-GTGCTTGTCACCTTGCAAAA-3′ (SEQ ID NO. 8), Gustducin: 5′-GAGAGCAAGGAATCAGCCAG-3′ (SEQ ID NO. 9) and 5′-GTGCTTTTCCCAGATTCACC-3′ (SEQ ID NO. 10), IL-25: 5′-ACAGGGACTTGAATCGGGTC-3′ (SEQ ID NO. 11) and 5′-TGGTAAAGTGGGACGGAGTTG-3′ (SEQ ID NO. 12), IL-33: 5′-CCTCCCTGAGTACATACAATGACC-3′ (SEQ ID NO. 13) and 5′-GTAGTAGCACCTGGTCTTGCTCTT-3′ (SEQ ID NO. 14), TSLP: 5′-CGTGAATCTTGGCTGTAAACT-3′ (SEQ ID NO. 15) and 5′-GTCCGTGGCTCTCTTATTCT-3′ (SEQ ID NO. 16), IL-17RB: 5′-ACCGTCTGTCGCTTCACTG-3′ (SEQ ID NO. 17) and 5′-CCACTTTATCTGCCGCTTGC-3′ (SEQ ID NO. 18).

Small intestine organoid culture and flow cytometry—Distal small intestinal organoids were prepared as previously described (Miyoshi et al., Nat Protoc. 8, 2471-2482 (2013)). To assess cytokine effects on tuft cells abundance, IL-13 (10 ng/ml, Biolegend, Endotoxin level <0.01 ng/μg) or IL-25 (50 ng/mil, Biolegend, Endotoxin level <0.01 ng/μg) were added to organoid media for 48 hours. To perform flow cytometry, organoids were liberated from the matrigel matrix as described by Miyoshi et al. and digested in DMEM containing 10% FBS, 0.5 units/ml Dispase II (StemCell Technologies), 50 μg/ml DNase (Roche) for 8 minutes at 37° C. The resulting solution was filtered through 40 μm mesh and stained for flow cytometry with APC-conjugated anti-EpCam (clone G8.8, Biolegend) with cell viability assessed with PI (Biolegend).

In vivo IL-25 injections-Gfilb^{EGFP/+} mice without parasites and Tritrichomonas muris colonized Gfilb^{EGFP/+}Trpm5^{-/-} mice were intraperitoneally (i.p.) injected daily with 0.5 μg of recombinant IL-25 (R&D, Endotoxin level <1.0 EU per 1 μg) or equivalent volume of sterile PBS for 7 days before harvesting the distal small intestine to examine the abundance of eosinophils and tuft cells as described above.

Statistical analyses—GraphPad Prism@ Software was used for the calculation of statistical measures, including mean values, standard errors, Shapiro-Wilk normality test, Mann-Whitney test, Ordinary one-way ANOVA, and Kruskal-Wallis test.

### Example 1: Evaluation DCLK1⁺Tuft Cells in the Distal Small Intestine of Wild Type (WT) Specific-Pathogen-Free Mice

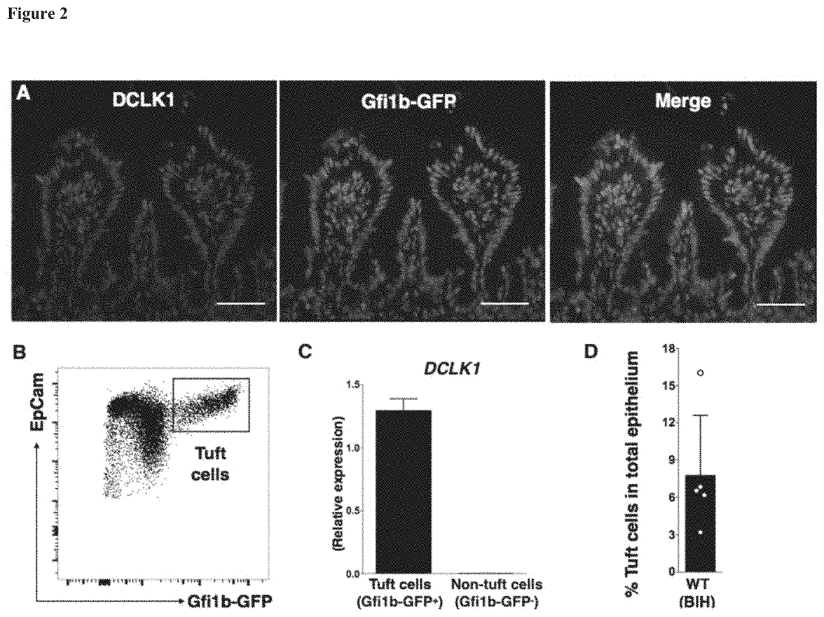

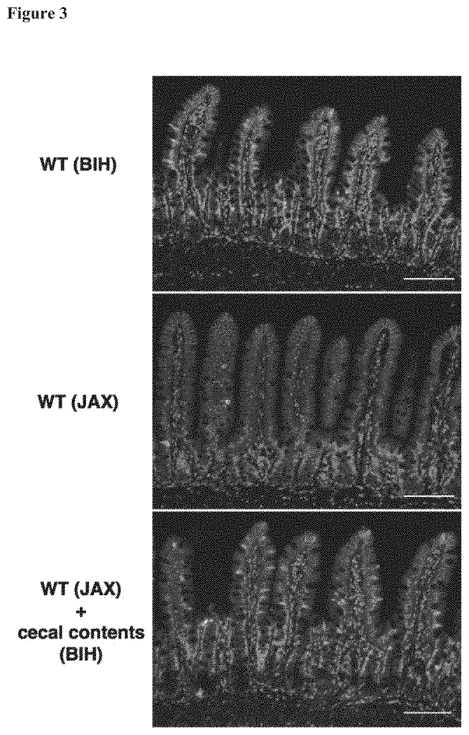

The frequency of DCLK1⁺ tuft cells in the distal small intestine of wild type (WT) specific-pathogen-free mice that were bred in-house (BIH) was evaluated. Markedly more intestinal DCLK1⁺ tuft cells (7.2%) (FIG. 1, Panel A) was found than previously known (0.4%) (19, 22) and this discrepancy was confirmed with an alternative tuft cell marker, Gfilb (23) (FIG. 2). As inter-institutional microbiota differences can contribute to substantial variation of mucosal immune cell populations, tuft cell abundance in mice obtained from Jackson laboratories (JAX) was compared to BIH mice. Tuft cells constituted 1.0% of the total IEC population of JAX mice (FIG. 1, Panel A). Feeding the cecal contents from BIH mice to JAX mice was sufficient to increase tuft cell populations to BIH levels (FIG. 3), suggesting that transmissible components of the BIH microbiota may drive tuft cell expansion when introduced to JAX mice. In support of this idea, intestinal histology revealed numerous single-celled protozoa in BIH but not JAX mice (FIG. 1, Panel B). To identify these protozoa, they were purified and imaged by scanning-electron microscopy (SEM) and identified as tritrichomonads (FIG. 1, Panel C). Quantitative-PCR confirmed that they were Tritrichomonas.

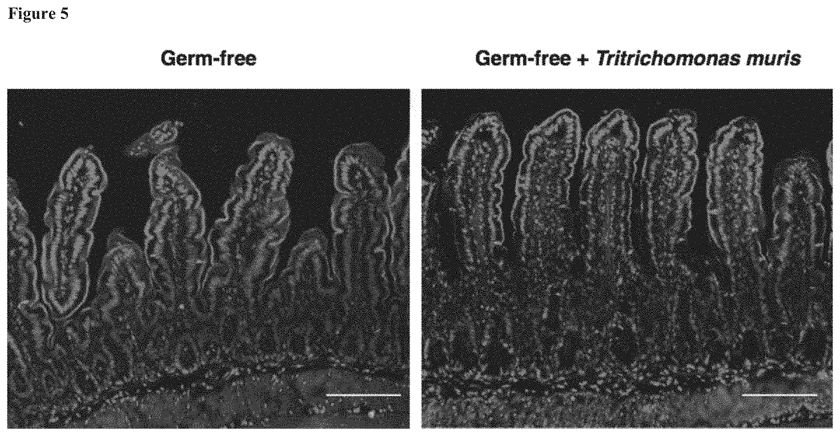

To eradicate T. muris from BIH mice, metronidazole (2.5 g/L) was added to their drinking water for 1 week. This eliminated T. muris and concomitantly reduced tuft cell abundance (FIG. 4). Because this treatment does not exclude the possibility that other metronidazole-sensitive organisms may contribute to tuft cell expansion, T. muris was cultured and colonized unexposed mice. T. muris colonization significantly elevated tuft cell numbers in conventional (FIG. 1, Panels E and F) and germ-free mice (FIG. 5), suggesting that this symbiotic protozoa is sufficient to increase tuft cell frequency.

### Example 2: Helminth Infection on Tuft Cell Abundance

To investigate the effect of helminth infection on tuft cell abundance, mice were infected with a diverse set of parasitic worms including: Heligmosomoides polygyrus (Hp), Trichinella spiralis (Ts), and Nippostrongylus brasiliensis (Nb). Similar to Example 1 with T. muris, infections with all three helminths increased tuft cell abundance, indicating that expansion of tuft cells is a broadly conserved feature of parasite colonization (FIG. 1, Panels G and H).

### Example 3—Upstream Pathways Mediate Tuft Cell Response

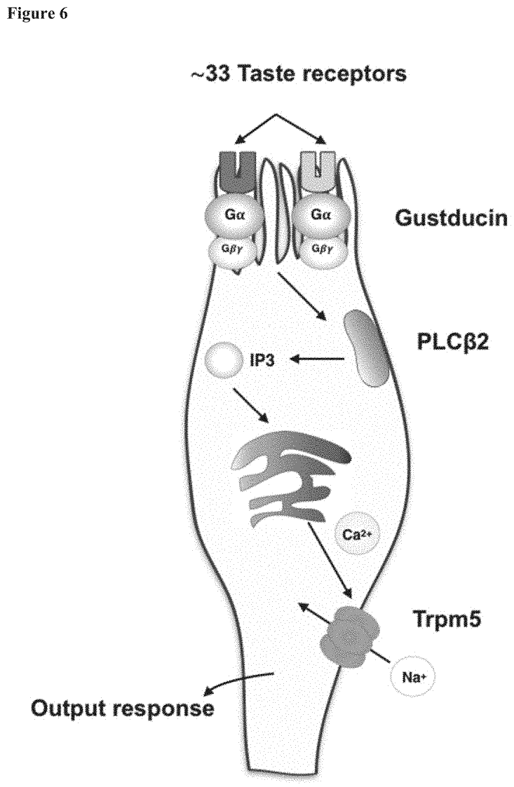

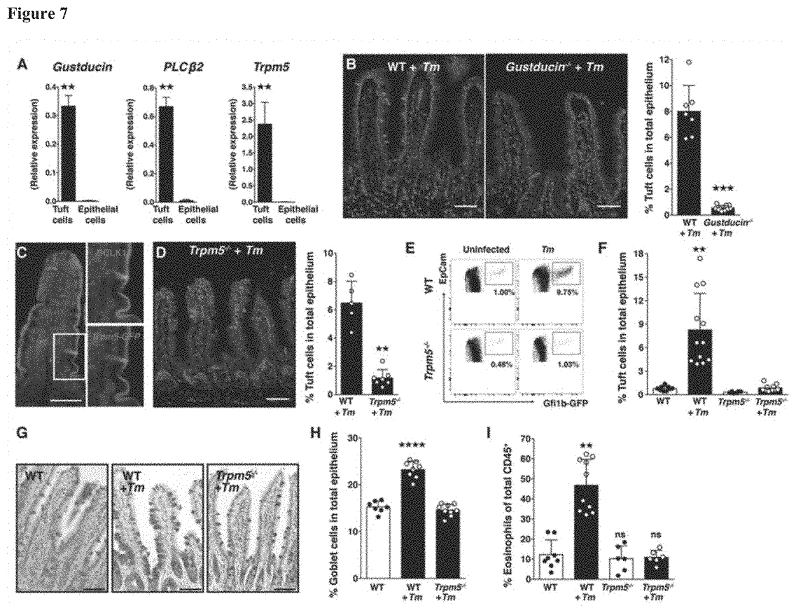

Because tuft cells are postulated to be chemosensory cells, whether perturbations to tuft chemosensory pathways may affect their expansion in response to parasites as well as the type 2 immune response typically initiated by parasites was considered. Multiple taste-GPCRs sense sweet, bitter, and umami compounds, yet engagement of these different receptors activates a common signal transduction pathway involving gustducin, PLCβ2, and Trpm5 (FIG. 6). Gfi1b⁺ tuft cells are the primary IEC subset expressing the canonical taste-associated components, gustducin, PLCβ2, and Trpm5 (FIG. 7, Panel A).

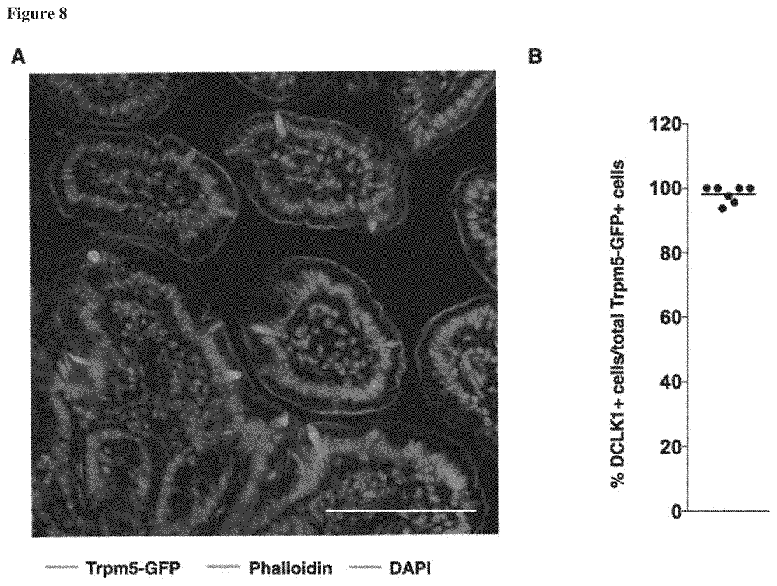

Tuft cell abundance in WT and gustducin^{-/-} mice colonized with T. muris was compared and significantly fewer tuft cells in gustducin^{-/-} animals (FIG. 7, Panel B) was found. Using Trpm5^{eGFP} reporter mice, Trpm5 is restricted to the epithelium and expressed by DCLK1+ tuft cells in the distal small intestine was validated (FIG. 7, Panel C and FIG. 8). Given the multiplicity of taste-GPCRs, the established role of Trpm5 in taste-chemosensation, and predominant intestinal Trpm5 expression by tuft cells, Trmp5-deficient mice were used to evaluate whether these pathways affect tuft cell parasite responses. Similar to gustducin^{-/-} mice, tuft cells failed to expand in Trpm5^{-/-} mice during T. muris colonization (FIG. 7, Panels D, E, and F). To determine if the blunted response was due to reduced parasite colonization, T. muris in the distal small intestine (FIG. 9) was measured. Slightly more parasites in both gustducin^{-/-} and Trpm5^{-/-} mice than WT (FIG. 9, Panel B) was found, indicating the lack of tuft cell response was not due to decreased T. muris colonization. Because T. muris is a stable component of the microbiota, how loss of Trpm5 would affect clearance of a pathogenic helminth such as H. polygyrus was queried. After 36 days post-infection, Trpm5^{-/-} mice had a significantly higher worm burden than WT (FIG. 9, Panel C). Collectively, these data suggest that pathways initiated upstream of Trpm5 may mediate tuft cell response to intestinal parasites.

Consistent with helminth infections, T. muris colonization also induced goblet cell hyperplasia in WT (P<0.0001), but not in Trpm5^{-/-} mice (FIG. 7, Panels G and H). Similarly, eosinophilia in WT but not Trpm5^{-/-} mice colonized with T. muris was observed (FIG. 7, Panel I).

### Example 4—Determination of TSLP, IL33, and Il25 Expression Patterns

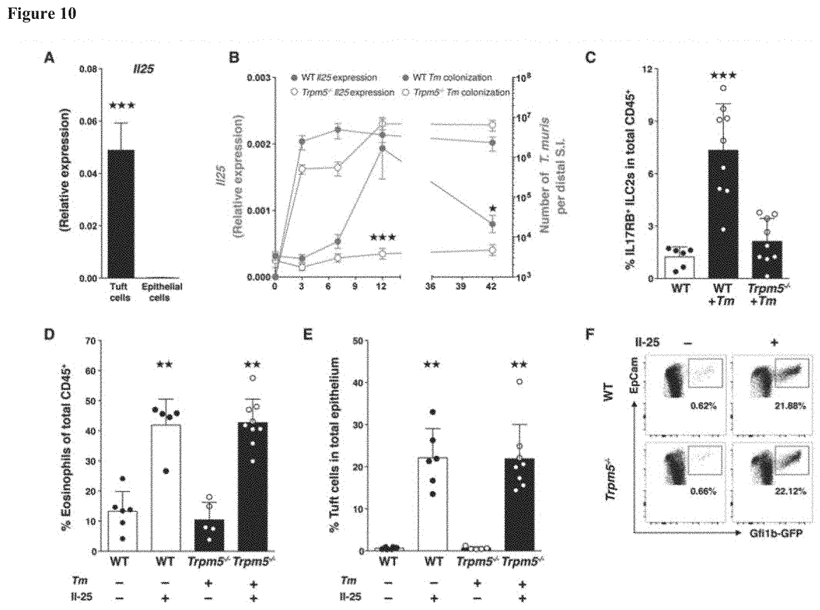

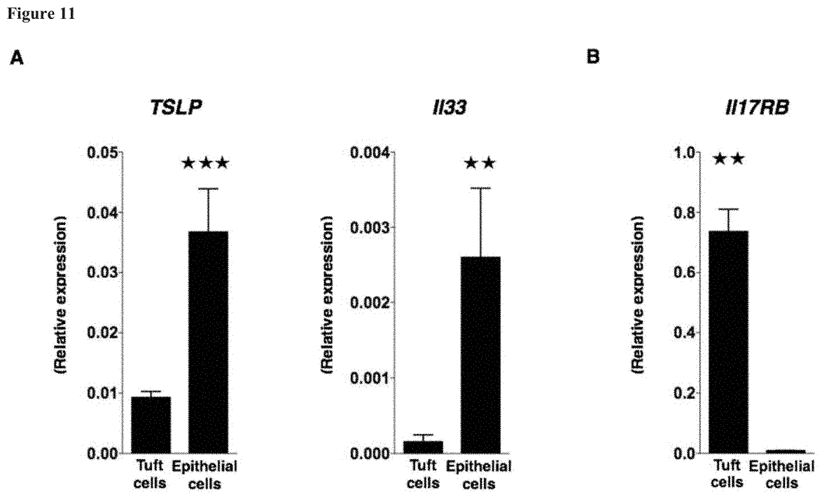

Because epithelial cells are a key source of the parasite-induced cytokines thymic stromal lymphopoietin (TSLP), interleukin-33 (IL-33) and interleukin-25 (IL-25), both tuft cells and the remaining epithelial fraction were isolated to determine TSLP, Il33, and Il25 expression patterns. Tuft cells expressed less TSLP and Il33 than other epithelial cells, and are the main source of epithelial Il25 (FIG. 10, Panel A and FIG. 11, Panel A). To determine if Trpm5 affects parasite-induced Il25 expression, WT and Trpm5^{-/-} mice were infected with T. muris and both parasite colonization and the corresponding epithelial Il25 expression were measured over time. T. muris rapidly colonized both WT and Trpm5^{-/-} mice, but Trpm5^{-/-} mice had significantly (P=0.0006) reduced Il25 expression 12 days post-infection (FIG. 10, Panel B).

IL-25 promotes proliferation and activation of type 2 innate lymphoid cells (ILC2s) via the receptor subunit, IL17RB. Accordingly, the frequency of intestinal lamina propria IL17RB⁺ ILC2s significantly increased in WT but not Trpm5^{-/-} mice after 12 days of T. muris infection (FIG. 10, Panel C). To determine if the parasite response in Trpm5^{-/-} mice could be complemented by exogenous IL-25, IL-25 intraperitoneally (i.p.) was injected into Trpm5^{-/-} mice and observed restoration of distal small intestinal eosinophilia and tuft cell abundance (FIG. 10, Panels D-F), suggesting that tuft cells may influence their own abundance.

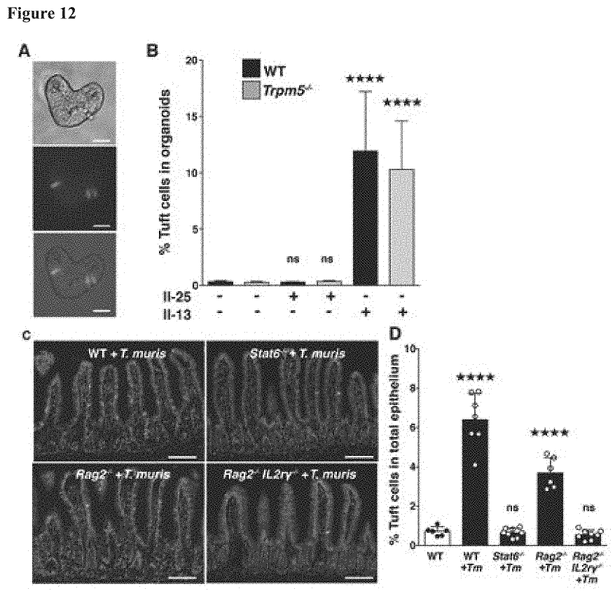

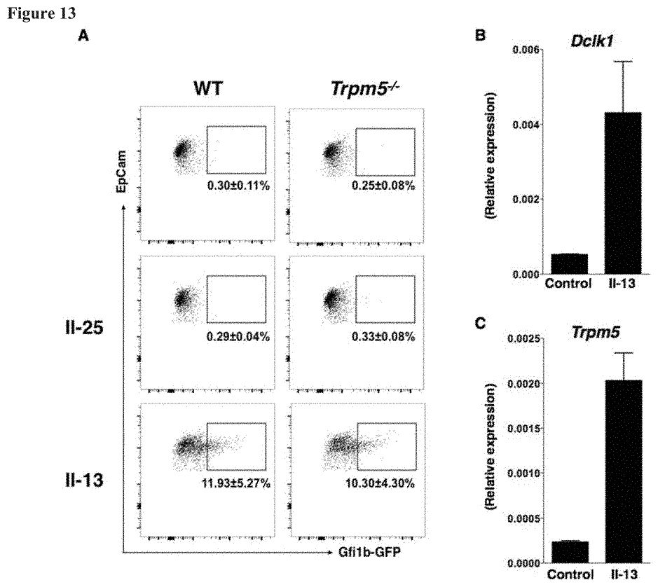

Epithelial cells are not only a crucial source of IL-25, but also signal in an autocrine manner via IL17RB. Therefore, tuft cell Il17RB expression was examined and found it was significantly higher (P=0.0043) than other epithelial cells (FIG. 11, Panel B). This raised the question of whether IL-25 induces tuft cell expansion via autocrine signaling or indirectly through recruitment of ILC2s. To evaluate factors that affect tuft cell abundance independently of the microbiota or immune system, an in vitro primary intestinal organoid system was employed. Small intestinal organoids reconstitute all the epithelial subsets from IEC stem cells and by generating organoids from Gfilb^{EGFP/+} mice, and GFP⁺ tuft cells (FIG. 12, Panel A and FIG. 13, Panel A) was detected. Both WT and Trpm5^{-/-} organoids contained approximately 0.3% tuft cells, but IL-25 did not increase tuft cell numbers (FIG. 10, Panel and FIG. 13, Panel A), suggesting that IL-25 does not act in an autocrine manner to expand tuft cell abundance. Since IL-25 promotes expansion of ILC2s, which are critical sources of IL-13 (11, 36, 40); a cytokine previously shown to increase goblet cell numbers, whether IL-13 may also increase tuft cell abundance was considered. IL-13 significantly expanded tuft cells from 0.3% of total organoid cells to 11.9% and 10.9% (WT and Trpm5^{-/-}, respectively) (FIG. 10, Panel B and Figure, 13, Panel A). In agreement with these results, expression of Dclk1 and Trpm5 also increased in IL-13 treated organoids (FIG. 13, Panels B and C).

### Example 5—Tuft Cells Detect T. muris Through Trpm5 Taste-Chemosensation

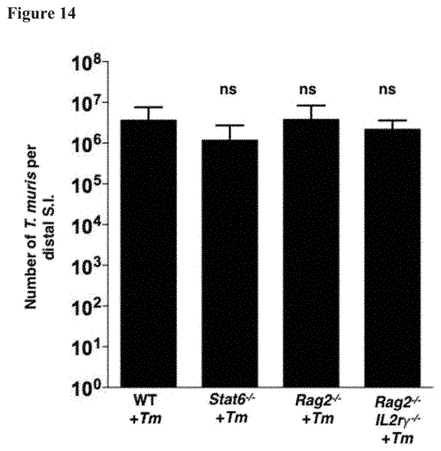

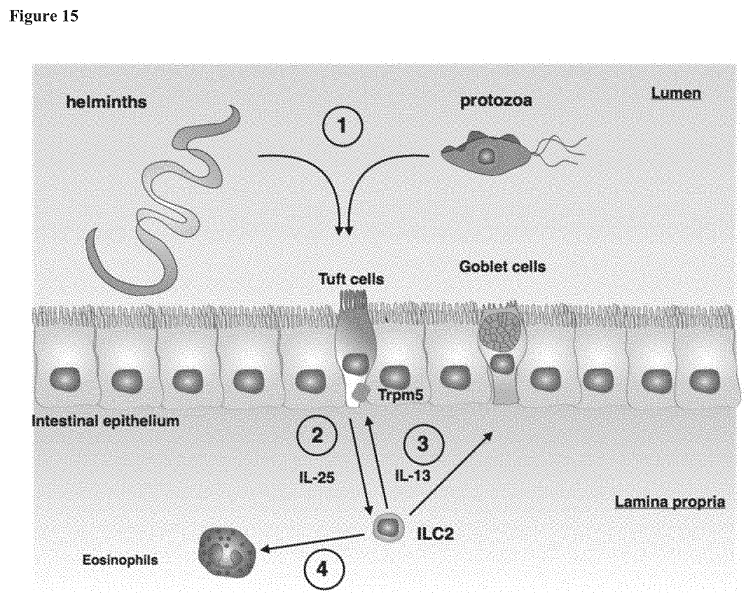

To determine if type 2 cytokine production by ILC2s may contribute to tuft cell expansion in vivo, WT, Stat6^{-/-}, Rag2^{-/-}, and Rag2^{-/-} I12rγ^{-/-} mice were colonized with T. muris (FIG. 14). STAT6 is activated by the type 2 cytokines, IL-4 and IL-13, and is required for intestinal helminth expulsion. Consistent with the organoid data demonstrating that IL-13 potently induces of tuft cell expansion, tuft cells did not expand when T. muris colonized Stat6^{-/-} mice (FIG. 12, Panels C and D). While both T helper 2 (T_{H}2) and ILC2 cells can produce IL-13 in mucosal tissue, parasite-induced IL-25 potently activates 1113 expression in ILCs. Tuft cell abundance was compared in Rag2^{-/-} mice which lack T_{H}2 cells but contain ILC2s and Rag2^{-/-} Il2rγ^{-/-} which lack both T_{H}2 and ILC2s cells. Infected Rag2^{-/-} mice had elevated tuft cell abundance compared to uninfected WT mice, yet similar to both Trpm5^{-/-} and Stat6^{-/-} mice, Rag2^{-/-} Il2rγ^{-/-} mice showed no tuft cell increase during T. muris infection (FIG. 12, Panels C and D). Collectively, this suggests that tuft cells may detect T. muris through Trpm5 taste-chemosensation to elicit ILCs, which in turn produce IL-13 to expand tuft cell abundance (FIG. 15).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of inducing a type 2 helper T cell (T_{H}2) immune response in a subject comprising administering to the subject a small molecule that enhances the activity or expression of Trpm5.

2. The method of claim 1, wherein the small molecule induces expression of IL-25 by the tuft cells.

3. The method of claim 1, wherein the small molecule induces expression of IL-13 by the subject.

4. The method of claim 1, wherein the small molecule is delivered orally to the subject.

5. The method of claim 1, wherein the subject has or is predisposed to an inflammatory bowel disease, and the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

6. The method of claim 1, wherein the subject has or is predisposed to a protozoan infection, wherein the protozoan infection is selected from leishmaniasis, trichomoniasis, trypanosomiasis (Chagas disease and sleeping sickness), toxoplasmosis, malaria, giardiasis, cryptosporidiosis, babesiosis, primary amoebic meningoencephalitis, amoebiasis, dientamoebiasis, rhinosporidosis, sarcocystosis, cyclosporiasis, isosporiasis, blastocystosis, balantidiasis, and granulomatous amoebic encephalitis.

7. The method of claim 1, wherein the subject has or is predisposed to a parasitic worm infection, wherein the parasitic worm is selected from coenurosis, diphyllobothriasis, echinococcosis, hymenolepiasis, taeniasis, cysticercosis, bertielliasis, sparganosis, schistosomiasis, clonorchiasis, fasciolosis, fasciolopsiasis, gnathostomiasis, metagonimiasis, paragonimiasis, opisthorchiasis, ascariasis, ancylostomiasis, angiostrongyliasis, baylisascariasis, filariasis, dracunculiasis, enterobiasis, halicephalobiasis, onchocerciasis, strongyloidiasis, thelaziasis, toxocariasis, trichinosis, trichuriasis, and elephantiasis.

8. A method of treating or preventing an inflammatory bowel disease in a subject comprising administering to the subject a small molecule that enhances the activity or expression of Trpm5.

9. The method of claim 8, wherein the small molecule induces expression of IL-25 by the tuft cells.

10. The method of claim 8, wherein the small molecule induces expression of IL-13 by the subject.

11. The method of claim 8, wherein the small molecule is delivered orally to the subject.

12. The method of claim 8, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

13. A method of treating or preventing a parasitic infection in a subject comprising administering to the subject a small molecule that enhances the activity or expression of Trpm5.

14. The method of claim 13, wherein the small molecule induces expression of IL-25 by the tuft cells.

15. The method of claim 13, wherein the small molecule induces expression of IL-13 by the subject.

16. The method of claim 13, wherein the small molecule is delivered orally to the subject.

17. The method of claim 13, wherein parasitic infection is a protozoan infection, and the protozoan infection is selected from leishmaniasis, trichomoniasis, trypanosomiasis (Chagas disease and sleeping sickness), toxoplasmosis, malaria, giardiasis, cryptosporidiosis, babesiosis, primary amoebic meningoencephalitis, amoebiasis, dientamoebiasis, rhinosporidosis, sarcocystosis, cyclosporiasis, isosporiasis, blastocystosis, balantidiasis, and granulomatous amoebic encephalitis.

18. The method of claim 13, wherein the parasitic infection is a parasitic worm infection, and the parasitic worm is selected from coenurosis, diphyllobothriasis, echinococcosis, hymenolepiasis, taeniasis, cysticercosis, bertielliasis, sparganosis, schistosomiasis, clonorchiasis, fasciolosis, fasciolopsiasis, gnathostomiasis, metagonimiasis, paragonimiasis, opisthorchiasis, ascariasis, ancylostomiasis, angiostrongyliasis, baylisascariasis, filariasis, dracunculiasis, enterobiasis, halicephalobiasis, onchocerciasis, strongyloidiasis, thelaziasis, toxocariasis, trichinosis, trichuriasis, and elephantiasis.
